(19) United States
(12) Patent Application Publication
(10) Pub. No.: US 2004/0106681 A1
(43) Pub. Date: Jun. 3, 2004

(54) Dosage escalation and divided daily dose of anti-depressants to treat neurological disorders

(75) Inventors: Srinivas G. Rao, San Diego, CA (US); Jay D. Kranzler, LaJolla, CA (US); R. Michael Gendreau, Poway, CA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Cypress Bioscience, Inc.

(21) Appl. No.: 10/678,767

(22) Filed: Oct. 3, 2003

Related U.S. Application Data
(60) Provisional application No. 60/415,739, filed on Oct. 3, 2002.
(60) Provisional application No. 60/431,550, filed on Dec. 6, 2002.
(60) Provisional application No. 60/443,081, filed on Jan. 28, 2003.
(60) Provisional application No. 60/443,203, filed on Jan. 28, 2003.

(51) Int. Cl. A61K 31/165

(52) U.S. Cl. 514/620

(57) ABSTRACT

The present invention provides a method to treat neurological disorders. The method includes, e.g., administering higher daily dosages of anti-depressant. The higher daily dosages result in an improved efficacy of the drug, the maintenance of a positive patient toleration, the maintenance of a positive patient safety profile (e.g., dose limiting toxicity), a suitable peak plasma concentration (Cₘₐₓ) of drug, and/or a once-a-day (QD) as opposed to twice-a-day (BID) administration. Applicant have discovered that increased daily dosages anti-depressant that would normally evoke adverse effects can be administered without the negative patient tolerability (i.e., adverse reactions) by escalating dosages over time. Such escalation dosages provide more efficacious amounts of anti-depressant than would otherwise be permitted. Similarly, higher levels of circulating drug are possible in patients by administering the compound in divided doses over the course of a day rather than once a day.

This application claims priority to U.S.S. No. 60/415,739 filed Oct. 3, 2002; U.S. S. No. 60/431,550 filed Dec. 6, 2002; U.S. S. No. 60/443,203 filed Jan. 28, 2003, and U.S. S. No. 60/443,081 filed Jan. 28, 2003.

### FIELD OF THE INVENTION

The invention is in the field of treating neurological disorders with an effective amount of anti-depressants such as the NSRI compound, milnacipran, administered in an escalating dosage to minimize undesirable side effects.

### BACKGROUND OF THE INVENTION

Neurological disorders such as Chronic Fatigue Syndrome, Fibromyalgia Syndrome, Chronic Pain, Depression Secondary to Pain and Functional Somatic Disorders will affect a large part of the population of the United States at some point in their lifetime. Although antidepressants are often used to treat many of these conditions, their effectiveness is often inadequate due to dose-limiting side effects. Most antidepressants are therefore restricted in their use because of adverse effects when they would otherwise be effective at treating symptoms of neurological disorders.

One class of antidepressants inhibit reuptake of the monoamines norepinephrine and serotonin and are termed serotonin-norepinephrine reuptake inhibitors (SNRIs). A subclass of these compounds preferentially inhibits norepinephrine reuptake with an equal or greater affinity than serotonin reuptake and are termed norepinephrin-serotonin reuptake inhibitors (NSRIs). Milnacipran (Z-2-aminomethyl-1-phenyl-N,N-diethylcyclopropane-carboxamide hydrochloride) is one such NSRI compound that preferentially inhibits norepinephrine reuptake over serotonin reuptake. Milnacipran is an approved and marketed drug in Europe for the treatment of depression. The regulatory dossier demonstrating the clinical efficacy of milnacipran in the treatment of depression is based on studies performed in Europe, USA, and Japan, including 5732 patients (4006 treated with milnacipran: 394 with placebo, 940 with TCAs and 344 with SSRIs). More than 30 double-blind trials have been performed comparing milnacipran either to placebo, tricyclic antidepressants (TCAs), or selective serotonin re-uptake inhibitors (SSRIs), involving both hospitalised and ambulatory patients with depression, as assessed by DSM III or RDC criteria (DSM III-R or DSM IV in the more recent studies).

These studies have shown that milnacipran is effective in major depressive episodes (adults and elderly) at a typical dose of 50 mg twice a day (BID) (taken with meals). At this dose, it has been shown that milnacipran exhibits:

a. Superior effectiveness to placebo: the meta-analysis of double-blind studies comparing milnacipran (50 mg BID) to placebo showed a significant difference between groups, both in Hamilton (HDRS or HAMD) and Montgomery-Asberg (MADRS) depression rating scales total scores;

b. The percentage of patient “responders” (i.e., patients whose HAMD or MADRS total scores decreased by 50% or more) was statistically superior to placebo, with 55% of all patients, and 64% of hospitalised patients responding to milnacipran, while 40% were responders to placebo;

c. Comparable effectiveness to TCAs: a response rate of 64% was seen with milnacipran, versus 67% with tricyclics (imipramine, amitriptyline and clomipramine); and

d. Comparable effectiveness to SSRIs: 64% response rate with milnacipran, versus 50-65% with SSRIs (fluvoxamine, fluoxetine and paroxetine).

Unfortunately, it is also known that daily dosages of greater than 50 mg of milnacipran are accompanied with negative patient tolerability (i.e., adverse reactions). It would be advantageous to administer daily dosages of greater than 50 mg of milnacipran without negative side effects.

Although anti-depressants such as milnacipran are effective in treating major depressive episodes and other neurological disorders, more suitable methods are needed to administer more effective amounts to treat these neurological disorders.

It is therefore an object of the present invention to provide a method of administering more efficacious amounts of anti-depressant to treat symptoms of neurological disorders.

It is a further object of this invention to provide a method to reduce dose limiting toxicity of a compound so that increased amounts of the compounds can be administered and maintain a positive patient safety profile.

It is a further object of this invention to provide a method to obtain a suitable peak plasma concentration (Cₘₐₓ) of anti-depressant by administering a dose once-a-day (QD), as opposed to twice-a-day (BID).

### SUMMARY OF THE INVENTION

A method to treat neurological disorders by administering higher daily dosages of anti-depressant is described where the side effects are minimized by escalating the dosage over a period of time. The higher daily dosages result in an improved efficacy of the drug, the maintenance of a positive patient toleration, the maintenance of a positive patient safety profile (e.g., dose limiting toxicity), a suitable peak plasma concentration (Cₘₐₓ) of drug, and/or a once-a-day (QD) as opposed to twice-a-day (BID) administration. Higher levels of circulating drug are also obtained by administering the compound in divided doses over the course of a day rather than once a day.

### BRIEF DESCRIPTION OF DRAWINGS

FIG. 1 is a flow chart showing the method of dosing patients with increasing weekly doses of milnacipran. Dose-limiting toxicity is evaluated at every dosage escalation throughout the study.

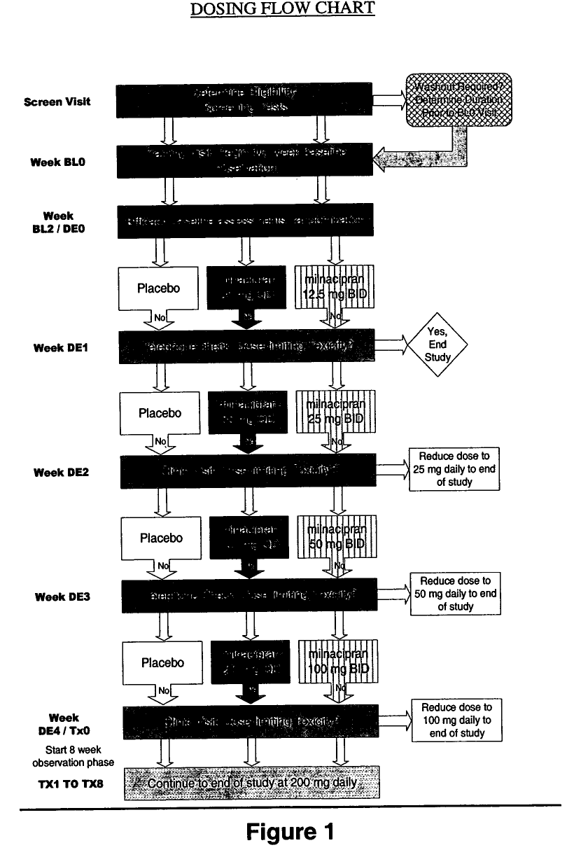

FIG. 2 is a plot of the percent of fibromyalgia syndrome (FMS) patients with improved, unchanged, or worsened global pain scores at the end of the 12-week treatment with milnacipran administered twice daily (BID) or once daily (QD) or with placebo.

FIG. 3 is a plot of the Beck depression scores of FMS patients who were diagnosed with major depression (MDE) at the baseline (before therapy) and at the endpoint of therapy with milnacipran BID or QD or with placebo.

FIG. 4 is a plot of the 24-hour daily pain reported scores of the three FMS treatment groups over the 12-week treatment period.

FIG. 5 is a plot of the self-reported daily sleep quality scores of patients in the three treatment groups over the 12-week treatment period.

FIG. 6 is a plot of the patient electronic diary pain scores (averaged for each patient over a one week period) of patients classified as responders over the 2-week baseline period and 12 weeks of treatment. RP=random prompt pain scores; Weekly=weekly recall pain score; Daily=daily recall score.

FIG. 7 is a plot of the change in hot plate latency period of rats pretreated with milnacipran injection, vehicle injection, or no injection in the swim stress test. The change plotted is the latency period measured after being subjected to the swim stress experience, sham swim experience, or no swimming (naive), minus the latency measured before being subjected to the experiences.

FIG. 8 is a plot of the change in grip strength of rats pretreated with milnacipran injection, vehicle injection, or no injection in the swim stress test. The change plotted is the grip strength measured after being subjected to the swim stress experience, sham swim experience, or no swimming (naive), minus the grip strength measured before being subjected to the experiences.

FIG. 9 is a plot of the hot plate latency period measured for rats after being subjected to the three swim stress test experiences and then, after the stress test experiences, being treated with milnacipran injection, vehicle injection, or no injection. The change plotted is the latency period measured after both (1) being subjected to the swim stress experience, sham swim experience, or no swimming (naive), and (2) being treated after the experiences with milnacipran, vehicle, or no injection, minus the grip strength measured on day 1, before the stress experiences.

FIG. 10 is a plot of the grip strength measured for rats after being subjected to the three swim stress test experiences and then, after the stress test experiences, being treated with milnacipran injection, vehicle injection, or no injection. The change plotted is the latency period measured after both (1) being subjected to the swim stress experience, sham swim experience, or no swimming (naive), and (2) being treated after the experiences with milnacipran, vehicle, or no injection, minus the grip strength measured on day 1, before the stress experiences.

Detailed Description of the Invention

Abbreviations

| | |
|---|---|
| DSP | Depression Secondary to Pain |
| CFS | Chronic Fatigue Syndrome |
| FMS | Fibromyalgia Syndrome |
| FSD | Functional Somatic Disorder |
| 5-HT | serotonin |
| NE | norepinephrine (noradrenaline) |
| NMDA | N-methyl D-aspartate |
| SNRIs | dual serotonin norepinephrine reuptake inhibitors, where |
| | serotonin reuptake exceeds norepinephrine reuptake. |
| NSRI | dual norepinephrine reuptake inhibitor where |
| | norepinephrine reuptake exceeds serotonin reuptake. |

I. Patients to be Treated.

Neurological disorders that can be treated with the escalating and/or divided dosage formulation include chronic pain, neuropathic pain, fibromyalgia syndrome, chronic fatigue syndrome, affective disorders/mood disorders, depression, a typical depression and functional somatic disorders. Symptoms of the neurological disorder can include but are not limited to musculoskeletal pain, fatigue, sleep disorder, sleep disturbance, or a combination thereof.

A neurological disorder is considered to be a chronic disorder when the patient has been afflicted with the disorder greater than 12 weeks but as early as six or even two weeks with persistent symptoms.

Chronic pain refers to pain that continues or recurs over a prolonged period of time (i.e., greater than three months), caused by various diseases or abnormal conditions, such as rheumatoid arthritis. Chronic pain may be less intense that acute pain. The person with chronic pain does not usually display increased pulse and rapid perspiration because the automatic reactions to pain cannot be sustained for long periods of time. Others with chronic pain may withdraw from the environment and concentrate solely on their affliction, totally ignoring their family, their friends, and external stimuli. See, Mosby's Medical, Nursing & Allied Health Dictionary, 5^{th} Edition (1998).

The chronic pain can be lower back pain, a typical chest pain, headache, pelvic pain, myofascial face pain, abdominal pain, and neck pain. Alternatively, the chronic pain can be caused by a disease or condition selected from the group of arthritis, temporal mandibular joint dysfunction syndrome, traumatic spinal cord injury, multiple sclerosis, irritable bowel syndrome, chronic fatigue syndrome, premenstrual syndrome, multiple chemical sensitivity, hyperventilation, closed head injury, fibromyalgia, rheumatoid arthritis, diabetes, cancer, HIV, and interstitial cystitis.

Similarly, neuropathic pain refers to pain associated with inflammation or degeneration of the peripheral nerves, cranial nerves, spinal nerves, or a combination thereof. The pain is typically sharp, stinging, or stabbing. The underlying disorder can result in the destruction of peripheral nerve tissue and can be accompanied by changes in the skin color, temperature, and edema. See, Mosby's Medical, Nursing & Allied Health Dictionary, 5^{th} Edition (1998); and Stedman's Medical Dictionary, 25^{th} Edition (1990).

Fibromyalgia syndrome (FMS) is a common systemic rheumatologic disorder estimated to affect 2-4% of the population, second in prevalence only to osteoarthritis. Fibromyalgia is associated with a reduced threshold for pain, generally related to pressure stimuli, and is often accompanied by fatigue, sleep disturbance, and morning stiffness. Other common symptoms include headache, migraine, non-cardiac chest pain, heartburn, palpitations, irritable bowel syndrome, variable bowel habit, diffuse abdominal pain, and urinary frequency. The diagnostic criteria for fibromyalgia require not only a history of widespread pain, but also the finding of tenderness on physical examination secondary to applied pressure In order to fulfill the criteria for fibromyalgia established in 1990 by the American College of Rheumatology (ACR), an individual must have both chronic widespread pain involving all four quadrants of the body as well as the axial skeleton, and the presence of 11 of 18 tender points on examination.

FMS is a medical problem reflecting a generalized heightened perception of sensory stimuli. The abnormality is thought to occur within the central nervous system (CNS) rather than peripherally, and the proposed pathophysiological defect is termed “central sensitization”. FMS patients typically suffer from both allodynia (perceiving pain even from a non-painful stimulus such as light touch) and hyperalgesia (an augmentation of pain processing in which a painful stimulus is magnified and perceived with higher intensity than it would be by a normal volunteer). In this regard, there are many parallels in the clinical presentation and proposed underlying mechanisms with neuropathic pain, such as diabetic neuropathy and trigeminal neuralgia.

Affective disorders/mood disorders are a variety of conditions characterized by a disturbance in mood as the main feature. If mild and occasional, the feelings may be normal. If more severe, they may be a sign of a major depressive disorder or dysthymic reaction or be symptomatic of bipolar disorder. Other mood disorders may be caused by a general medical condition. See, Mosby's Medical, Nursing & Allied Health Dictionary, 5^{th} Edition (1998).

Depression is an abnormal mood disturbance characterized by feelings of sadness, despair, and discouragement. Depression refers to an abnormal emotional state characterized by exaggerated feelings of sadness, melancholy, dejection, worthlessness, emptiness, and hopelessness, that are inappropriate and out of proportion to reality. See, Mosby's Medical, Nursing & Allied Health Dictionary, 5^{th} Edition (1998).

The depression can be at least one of a major depressive disorder (single episode, recurrent, mild, moderate, severe without psychotic features, severe with psychotic features, chronic, with catatonic features, with melancholic features, with a typical features, with postpartum onset, in partial remission, in full remission), dysthymic disorder, adjustment disorder with depressed mood, adjustment disorder with mixed anxiety and depressed mood, premenstrual dysphoric disorder, minor depressive disorder, recurrent brief depressive disorder, postpsychotic depressive disorder of schizophrenia, a major depressive disorder associated with Parkinson's disease, and a major depressive disorder associated with dementia.

Depression secondary to pain (DSP) is a depressive disorder characterized by the co-morbidity of pain and a typical depression. Specifically, the pain can be chronic pain, neuropathic pain, or a combination thereof. The DSP can include a typical depression and chronic pain where the chronic pain precedes the a typical depression or where the a typical depression precedes the chronic pain. Alternatively, the DSP includes a typical depression and neuropathic pain.

The atypical depression can include mood reactivity and two or more neurovegetative symptoms present for more than about two weeks such as hypersomnia, increased appetite or weight gain, leaden paralysis, and a long standing pattern of extreme sensitivity to perceived interpersonal rejection.

Atypical depression is a depressed affect, with the ability to feel better temporarily in response to positive life effect (mood reactivity), plus two or more neurovegetative symptoms present for more than about two weeks selected from the group of hypersomnia, increased appetite or weight gain, leaden paralysis, and a long standing pattern of extreme sensitivity to perceived interpersonal rejection. Those of skill in the art recognize that the neurovegetative symptoms can be reversed compared to those found in other depressive disorders (e.g., melancholic depression); hence the term “atypical.”

Functional somatic disorder (FSD) refers to several related syndromes typically characterized by symptoms, suffering and disability rather than by disease-specific abnormalities of tissue structure or function. Patients with functional somatic syndrome are physically healthy, but encounter disabling, medically unexplained symptoms. Symptoms include complaints such as fatigue, headache, joint pains, weakness, memory problems, anxiety and palpitations. Common functional somatic syndromes include multiple chemical hypersensitivity, sick building syndrome, repetition stress injury, chronic whiplash, chronic Lyme disease, the side effects of silicone breast implants, candidiasis sensitivity, Gulf War syndrome, mitral valve prolapse and hypoglycemia.

Patients with functional somatic disorder typically provide themselves with self-diagnoses for their complaints and resist information that contradicts attribution of their symptoms to a specific disease. These patients have a higher incidence of psychiatric disorders, particularly, anxiety, depressive and somatoform disorders. Psychosocial factors that amplify symptoms of the patient include the belief that the patient has a serious disease; the expectation of the patient that the condition will likely worsen; the “sick role” including the effects of litigation and compensation; and the alarming portrayal by the patient that the condition is catastrophic and disabling. Nonetheless, patients diagnosed with functional somatic disorder are characterized by considerable suffering and disability.

Functional somatic disorder can also be associated with pain. The pain can either precede or follow the development of functional somatic disorder and the pain can be chronic pain or neuropathic pain or a combination of the two.

II. Compositions

A. Active Compounds

The active compounds used in this method possess anti-pain or analgesic activity, anti-depressant activity and are therefore useful as agents for the treatment of pain, depression and related diseases and symptoms.

The compounds used in this method are also useful as standard or reference compounds for use in tests or assays for determining the ability of an agent to treat, prevent, or lessen the conditions or symptoms associated a neurological disorder, for example in a pharmaceutical research program. Thus, the compounds disclosed herein may be used as control or reference compound in such assays and as a quality control standard. The compounds of the present invention may be provided in a commercial kit or container for use as such standard or reference compound.

Norepinephrine (NE)—serotonin (5-HT) reuptake inhibitors (NSRIs) refers to a class of compounds that inhibits the reuptake of both norepinephrine (NE) and serotonin (5-HT), but preferentially blocks the reuptake of NE over that of 5-HT. The selective NSRI will have an NE: 5-HT reuptake inhibition ratio of at least about 1. Specifically, the selective NSRI can have an NE:5-HT reuptake inhibition ratio of up to about 50. More specifically, the selective NSRI can have an NE:5-HT reuptake inhibition ratio of about 1:1 to about 20:1. More specifically, the selective NSRI can have an NE:5-HT reuptake inhibition ratio of about 1:1 to about 5:1. More specifically, the selective NSRI can have an NE:5-HT reuptake inhibition ratio of about 1:1 to about 3:1.

An NSRI has an IC₅₀ for 5-HT reuptake of 200 nM or less and an IC₅₀ for NE reuptake of 200 nM or less, and an IC₅₀ for dopamine reuptake of at least 1000 nM. The NSRI will have an NE:5-HT reuptake inhibition ratio of at least about 0.5:1. The NE:5-HT reuptake inhibition ratio is calculated by dividing the IC₅₀ for 5-HT reuptake by the IC₅₀ for NE reuptake. For instance, if a compound has an IC₅₀ for NE reuptake of 10 nM and an IC₅₀ for 5-HT reuptake of 20 nM, it has an NE:5-HT reuptake inhibition ratio of 2:1. In specific embodiments, the NSRI will have an NE:5-HT reuptake inhibition ratio of about 0.5:1 to about 50:1, about 1:1 to about 20:1, about 0.5:1 to 5:1, about 1:1 to about 5:1, about 0.5:1 to about 3:1, or about 1:1 to about 3:1.

The NSRI compounds can exhibit antagonist properties at the NMDA receptor. An NMDA receptor antagonist binds to and decreases the activity of an NMDA receptor. This includes both non-competitive and competitive NMDA receptor antagonists, glycine-site antagonists, glutamate antagonists, and allosteric antagonists. A compound can be determined to be an NMDA receptor antagonist by assays known to those of skill in the art.

“Milnacipran” (±)-cis-2-(aminomethyl)-N,N-diethyl-1-phenylcyclopropanecarboxamide hydrochloride (CAS Registry Number is 92623-85-3) is an NSRI compound of the formula:

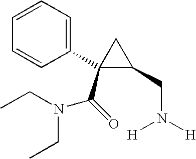

Methods of preparing milnacipran are disclosed, e.g., in U.S. Pat. No. 4,478,836 and references cited therein. Unless otherwise indicated, milnacipran can include all sterioisomeric forms, mixtures of sterioisomeric forms, and pharmaceutically acceptable salts thereof.

It is believed that that the dextrogyral enantiomer of milnacipran is about twice as active in inhibiting norepinephrine and serotonin reuptake than the racemic mixture, and that the levrogyral enantiomer is much less potent. See, e.g., Viazzo et al., 1996, Tetrahedron Lett. 37(26):4519-4522; Deprez et al., 1998, Eur. J. Drug Metab. Pharmacokinet. 23(2): 166-171). Accordingly, milnacipran can be administered in enantiomerically pure form (e.g., the pure dextrogyral enantiomer) or as a mixture of dextrogyral and levrogyral enantiomers, such as a racemic mixture.

When required, separation of the racemic mixture of milnacipran can be achieved by HPLC using a chiral column or by a resolution using a resolving agent such as camphonic chloride as in Thomas J. Tucker, et al., J. Med. Chem. 1994 37, 2437-2444. Milnacipran may also be directly synthesized using a chiral catalyst or a chiral ligand, e.g. Mark A. Huffman, et al., J. Org. Chem. 1995, 60, 1590-1594.

Known adverse reactions to oral administration of milnacipran can include nausea, vomiting, headache, tremulousness, anxiety, panic attack, palpitations, urinary retention, orthostatic hypotension, diaphoresis, chest pain, rash, weight increase, back pain, constipation, diarrhea, vertigo, increased sweating, agitation, hot flushes, tremors, fatigue, somnolence, dyspepsia, dysoria, nervousness, dry mouth, abdominal pain, insomnia, or a combination thereof.

Other SNRIs which can be administered using dosage escalation and/or divided dosages include the following:

Venlafaxine hydrochloride ((R/S)-1-[2-(demethylamino)-1-(4-methoxyphenyl)ethyl] cyclohexanol hydrochloride) or ((±)-1-[α[(dimethylamino)methyl]-p-methoxybenzyl]cyclohexanol hydrochloride);

Mirtazapine (1,2,3,4,10,14b-hexahydro-2-methylpyrazino[2,1-a]pyrido[2,3-c]benzazepine);

Nefazodone hydrochloride (2-[3-[4-(3-chlorophenyl)-1-piperazinyl]propyl]-5-ethyl-2,4-dihydro-4-(2-phenoxyethyl)-3H-1,2,4-triazol-3-one monohydrochloride);

Thioridazine hydrochloride (10H-Phenothiazine, 10-[2-(1-methyl-2-piperidinyl)ethyl]-2-(methylthio)-, monohydrochloride);

Bupropion hydrochloride ((±)-1-(3-chlorophenyl)-2-[(1,1-dimethylethyl)amino]-1-propanone hydrochloride);

Monoamine oxidase inhibitors such as:

Tranylcypromine sulfate ((±)-trans-2-phenyl-cyclopropylamine sulfate (2:1));

Phenelzine sulfate (Phenethylhydrazine hydrogen sulphate);

Moclobemide (p-chloro-N-(2-morpholinoethyl)benzamide);

Pirlindole (2,3,3a,4,5,6-Hexahydro-8-methyl-1H-pyrazino[3,2,1-j,k]carbazole;

Selective Serotonin reuptake inhibitors such as:

Citalopram hydrobromide ((±)-1-(3-dimethylaminopropyl)-1-(4-fluorophenyl)-1,3-dihydroisobenzofuran-5-carbonitrile, HBr);

Paroxetine hydrochloride ((−)-trans-4R-(4′-fluorophenyl)-3S-[(3′,4′-methylenedioxyphenoxy)methyl]piperidine hydrochloride hemihydrate);

Fluoxetine hydrochloride ((±)-N-methyl-3-phenyl-3-[(α,α,α-trifluoro-p-tolyl)oxy]propylamine hydrochloride);

Sertraline hydrochloride ((1S-cis)-4-(3,4-dichlorophenyl)-1,2,3,4-tetrahydro-N-methyl-1-naphthalenamine hydrochloride);

Tricyclic Antidepressants such as:

Amitriptyline HCl (3-(10,11-dihydro-5H-debenzo [a,d]cycloheptene-5-ylidene)-N,N-dimethyl-1-propanamine hydrochloride).

Desipramine hydrochloride (5H-Dibenz[bf]azepine-5-propanamine, 10,11-dihydro-N-methyl-, monohydrochloride);

Doxepin hydrochloride (1-Propanamine, 3-dibenz[b,e]oxepin-11(6H)ylidene-N,N-dimethyl-, hydrochloride);

Trimipramine maleate (5-(3-dimethylamino-2-methylpropyl)-10,11-dihydro-5H-dibenz (b,f) azepine acid maleate (racemic form));

Protriptyline HCl (N-methyl-5H-dibenzo[a,d]-cycloheptene-5-propanamine hydrochloride);

Anti-covulsants such as:

Divalproex sodium (sodium hydrogen bis(2-propylpentanoate));

Clonazepam (5-(2-chlorophenyl)-1,3-dihydro-7-nitro-2H-1,4-benzodiazepin-2-one); and

Alprazolam (8-Chloro-1-methyl-6-phenyl-4H-s-triazolo[4,3-α][1,4]benzodiazepine);

The above compounds can be substantially free of bodily fluids. Additionally, these compounds can be at least 90 wt. % pure, at least 95 wt. % pure, at least 98 wt. % pure or at least 99 wt. % pure.

B. Salts & Derivatives

Pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

Examples of pharmaceutically acceptable salts include, but are not limited to, mineral or organic acid salts of basic residues such as amines and alkali. The pharmaceutically acceptable salts include the conventional non-toxic salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic. Specifically, the pharmaceutically acceptable salt can be the hydrochloric or hydrochloride (HCl) salt.

These compounds are suitable for use in contact with the tissues of human beings and animals without excessive toxicity, irritation, allergic response, or other problem or complication commensurate with a reasonable benefit/risk ratio.

Suitable compounds also include prodrugs and metabolites of the active ingredients. Prodrugs are any covalently bonded substances which release the active parent drug or other formulas or compounds of the present invention in vivo when such prodrug is administered to a subject.

A metabolite is any substance resulting from biochemical processes by which living cells interact with the active compound and includes products or intermediates from any metabolic pathway.

C. Combinations of Active Ingredients

Each therapeutic agent used in this method of treatment can independently be in any dosage form and can also be administered in combination. The agents may be formulated together, in a single dosage unit (that is, combined together in one capsule, tablet, powder, or liquid, etc.) as a combination product and administered at the same time as a single compound or in any order if not formulated together in a single dosage unit. Preferably the agents are administered within one hour of each other if administered separately.

The proper dosage of co-administered agents will be readily ascertainable by a medical practitioner skilled in the art, based upon the present disclosure. By way of general guidance, typically a daily dosage may be about 100 milligrams to about 1.5 grams of each component. If more than one compound is administered, then typically a daily dosage may be about 100 milligrams to about 1.5 grams of each agent. By way of general guidance, when multiple agents are administered in combination, the dosage amount of each agent may be reduced by about 70-80% relative to the usual dosage when it is administered alone in view of the synergistic effect of the combination.

D. Formulations and Excipients

Formulations

The active ingredient can be administered orally in solid dosage forms, such as capsules, tablets and powders, or in liquid dosage forms, such as elixirs, syrups and suspensions. It can also be administered parenterally, in sterile liquid dosage forms. Additives may also be included in the formulation to enhance the physical appearance, improve stability, and aid in disintegration after administration. For example, liquid dosage forms for oral administration can contain coloring and flavoring to increase patient acceptance.

The daily dosages of active ingredient can be administered in a once-a-day (QD) dosage or alternatively in a divided dosage, e.g., a twice-a-day (BID) dosage.

Gelatin capsules contain an active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours or days and can also be formulated for implantation or transdermal/transmucosal delivery. Such formulations typically will include a polymer that biodegrades or bioerodes thereby releasing a portion of milnacipran. The formulations may have the form of microcapsules, liposomes, solid monolithic implants, gels, viscous fluids, discs, or adherent films.

Compressed tablets can be sugar coated or film coated to mask any unpleasant taste and protect the tablet from the atmosphere, or enteric coated for selective disintegration in the gastrointestinal tract.

Kits

The active ingredients can be formulated as kits which include divided daily dose equivalents or doses of increasing concentration combined with a container for holding the dosage units and printed indica with instructions for administering the doses.

Preferably the kit used for a 4-step dosage escalation includes a first unit dosage of active ingredient up to 100 mg. A second dosage amount is included that is 1.5 to 2.5 times greater than the first dosage amount. A third dosage amount is included that is 1.5 to 2.5 times greater than the second dosage amount. A fourth dosage amount is included that is 1.5 to 2.5 times greater than the third dosage amount. This kit also contains a container for holding the four dosage unit forms and printed indicia with instructions for administering the dosages. The first unit dosage form can be in the form of a once-a-day (QD) dosage. This kit format can be modified to include more or fewer dosage units of increasing dosage amount.

A preferred embodiment of the kit includes a first dosage amount of milnacipran of up to 100 mg and a second dosage unit of milnacipran greater than 100 mg. This kit also contains a container for holding the two dosage unit forms and printed indicia with instructions for administering the dosages. This kit can be modified to include four escalating dosage amounts of milnacipran (1) 20-30 mg, (2) 40-60 mg, (3) 75-125 mg and (4) 175-225 mg along with the container for holding the dosage unit forms and instructions for administering the dosages.

Excipients

In addition to the active or therapeutic ingredient, tablets contain a number of inert materials. The latter are known as additives or “adds.” They may be classified according to the part they play in the finished tablet. The first group contains those which help to impart satisfactory compression characteristics to the formulation. These include (1) diluents, (2) binders, and (3) lubricants. The second group of added substances helps to give additional desirable physical characteristics to the finished tablet. Included in this group are (1) disintegrators, (2) colors, and in the case of chewable tablets, (3) flavors, and (4) sweetening agents.

Frequently the single dose of active ingredient is small and an inert substance is added increase the bulk in order to make the tablet a practical size for compression. Diluents used for this purpose include dicalcium phosphate, calcium sulfate, lactose, kaolin, mannitol, sodium chloride, dry starch, and powdered sugar based on compatability between the diluant and the active ingredient.

Agents used to impart cohesive qualities to the powdered material are referred to as binders or granulators. They impart a cohesiveness to the tablet formulation which insures the tablet remaining intact after compression, as well as improving the free-flowing qualities by the formulation of granules of desired hardness and size. Materials commonly used as binders include starch, gelatin, and sugars as sucrose, glucose, dextrose, molasses, and lactose. Natural and synthetic gums which have been used include acacia, sodium alginate, extract of Irish moss, panwar gum, ghatti gum, mucilage of isapol husks, carboxymethylcellulose, methylcellulose, polyvinylpyrrolidone, Beegum, and larch arabogalactan. Other agents which may be considered binders under certain circumstances are polyethylene glycol, ethylcellulose, waxes, water and alcohol.

Lubricants are used in tablet manufacture to improve the rate of flow of the tablet granulation, prevent adhesion of the tablet material to the surface of the dies and punches, reduce interparticle friction, and facilitate the ejection of the tablets from the die cavity. Commonly used lubricants include talc, magnesium stearate, calcium stearate, stearic acid, and hydrogenated vegetable oils.

A disintegrator is a substance, or a mixture of substances, added to a tablet to facilitate its breakup or disintegration after administration. Materials serving as disintegrates have been chemically classified as starches, clays, celluloses, aligns, or gums.

In addition to the starches a large variety of materials have been used and are reported to be effective as disintegrators. This group includes Veegum HV, methylcellulose, agar, bentonite, cellulose and wood products, natural sponge, cation-exchange resins, alginic acid, guar gum, citrus pulp, and carboxymethylcellulose. Sodium lauryl sulfate in combination with starch also has been demonstrated to be an effective disintegrant.

The non-aqueous carrier, or excipient, can be any substance that is biocompatible and liquid or soft enough at the mammal's body temperature to release the active ingredient into the bloodstream at a desired rate. The carrier is usually hydrophobic and commonly organic, e.g., an oil or fat of vegetable, animal, mineral or synthetic origin or derivation. Preferably, the carrier is immiscible in water and/or soluble in the substances commonly known as fat solvents.

Typically, water, suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of milnacipran, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are known in the art.

III. Methods of Use

A. Treatment Protocol

The administration of active compound should be at an effective amount to treat symptoms of the neurological disorder while being substantially free of adverse reactions to the patient (meaning a decrease in, or lack of, abnormal, harmful, or unintended reaction to a drug (e.g., milnacipran)). The term “substantially free of adverse reactions” is relative to the number, nature and degree of adverse reactions of a specified dosage of an active ingredient. The active compound should be administered for a therapeutically effective period of time to ameliorate or eliminate symptoms of the neurological disorder.

The adverse reaction can be associated with at least one of the following: skin, central and peripheral nervous system, vision, psychiatric, gastrointestinal system, liver and biliary system, endocrine and metabolic system, cardiovascular system, respiratory system, red blood cells, white blood cells, platelets, blood, urinary system, reproductive system, and neoplasms. The adverse reaction can include at least one of the following: nausea, vomiting, headache, tremulousness, anxiety, panic attack, palpitations, urinary retention, orthostatic hypotension, diaphoresis, chest pain, rash, weight increase, back pain, constipation, vertigo, increased sweating, agitation, hot flushes, tremors, fatigue, somnolence, dyspepsia, dysoria, nervousness, dry mouth, abdominal pain, and insomnia.

The compounds can be administered alone, but preferably are administered with a pharmaceutical carrier selected on the basis of the chosen route of administration and standard pharmaceutical practice. They can be administered by any conventional means available for use in conjunction with pharmaceuticals, either as individual therapeutic agents or in a combination of therapeutic agents.

Preferably the daily dosage of the active ingredient is escalated over a period of time to achieve a therapeutic amount of circulating active compound in the patient (e.g., first period of time and/or second period of time).

The gradual escalation of daily dosage is intended to improve tolerance of the patient to the active ingredient administered. Meaning that the patient treated with the active ingredient will: (1) not experience an adverse reaction; (2) experience less adverse reactions; (3) experience adverse reactions of a lesser degree in severity; or a combination thereof. This tolerability is relative to the tolerance of a mammal to the active ingredient at a specified dosage, as compared to the tolerance of a mammal to that same dosage of active ingredient in the absence of a dose escalation procedure.

Generally, the active compound can be administered in increasing dosage amounts in a stepwise manner to increase the circulating dosage of the active compound with increasing patient tolerance to avoid or minimize adverse reactions. The daily dosage of active compound can vary during each period of time (i.e. each step) provided each of the dosage amounts is within the allotted dosage range for that step.

For example in a 4 step escalation, the active compound can be administered in a daily dosage of up to 100 mg for a first period of time and then escalated up to 1.5-2.5 times the amount of the initial dosage amount for a second period of time. The second period of time can be of a similar duration as the first period of time after which the dosage of active ingredient is escalated again to 1.5-2.5 times the amount of the second dosage amount for a third period of time. The third period of time can be a similar duration to the first and second periods of time after which the dosage of active ingredient is escalated again to 1.5-2.5 times the amount of the third dosage amount for a therapeutically effective period of time to treat symptoms of the neurological disorder. Alternatively, in a 2 step escalation, the dosage can be escalated only once to a second dosage amount and the second period of time can be a therapeutically effective amount of time to treat symptoms of the neurological disorder.

The periods of time for each step in the dosage escalation can be 3 days long, greater than 3 days, or greater than 2, 4, 6, 8, 10, 12 or 20 weeks. The daily dosage can be administered once or can be divided up two or more (e.g. 2, 3, 4, or 5) times a day.

One embodiment entails a 2-step escalation by administering a daily dosage of milnacipran of up to 100 mg for greater than 3 days and then escalating the daily dosage amount to greater than 100 mg for a therapeutically effective amount of time to treat symptoms of the neurological disorder.

Another embodiment entails a 3-step escalation by administering milnacipran at an initial daily dosage amount between about 10 and 50 mg for greater than 3 days and then escalating the daily dosage amount to about 25-75 mg for greater than 3 days and then escalating the daily dosage amount to greater than 100 mg for a therapeutically effective amount of time to treat symptoms of the neurological disorder.

Another embodiment entails a 4-step escalation by administering milnacipran at an initial daily dosage amount between about 20 and 30 mg for 7 days and then escalating the daily dosage amount to about 40-60 mg for 7 days and then escalating the daily dosage amount to about 75-125 mg for 7 days and then escalating the daily dosage amount to escalating the daily dosage amount to 175-225 mg for a therapeutically effective amount of time to treat symptoms of the neurological disorder.

These administration protocols can be applied to any of the anti-depressant compounds described above.

B. Effective Dosage Ranges.

The dosage administered will vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to about 1000 milligrams per kilogram of body weight, with the preferred dose being about 0.1 to about 100 mg/kg, preferably administered several times a day.

The dosages of active ingredient (e.g. milnacipran) disclosed herein possess suitable activity in treating symptoms of neurological disorders such as: (1) an improved efficacy of active ingredient, (2) the maintenance of a positive patient toleration, (3) the maintenance of a positive patient safety profile (e.g., dose limiting toxicity), (4) a suitable peak plasma concentration (Cₘₐₓ) of active ingredient, and/or (5) a once-a-day (QD) as opposed to twice-a-day (BID) administration.

An NSRI antidepressant can be administered in a daily dosage greater than (e.g., about 1.5 times to about 4.0 times greater than) the recommended daily dosage of the anti-depressant to treat depression. The recommended daily dosages for the anti-depressants disclosed herein, to treat depression, can be found, e.g., in Physician's Desk Reference (PDR), 55^{th} Edition (2001); and the Internet Drug Index website (www.RxList.com); or the SLS Psychiatric and Associated Drugs website (http://sl.schofield3.home.att.net/medicine/psychiatric_drugs_chart.html).

The NSRI anti-depressant can be administered in more divided dosages than is recommended to treat depression when treating symptoms of other neurological. The recommended daily dosages for the anti-depressants, to treat depression, can be found, e.g., in Physician's Desk Reference (PDR), 55^{th} Edition (2001); the Internet Drug Index website (www.RxList.com); or the SLS Psychiatric and Associated Drugs website (http://sl.schofield3.home.att.net/medicine/psychiatric_drugs_chart.html).

Generally, in a 2-step dosage escalation, the first dosage amount is up to about 100 mg per day and the second dosage amount is greater than about 100 mg per day. Preferably the dosage of the next escalation step is generally 1.5 to 2.5 times the amount of the previous dosage. Thus for a 4 step escalation protocol, the second daily dosage is about 1.5 times to about 2.5 times of the first daily dosage; the third daily dosage is about 1.5 times to about 2.5 times of the second daily dosage; and the fourth daily dosage is about 1.5 times to about 2.5 times of the third daily dosage administered for a sufficient period of time to effectively treat the symptoms of the neurological disorder.

For example, a preferable dosage regime for a 3-step dosage escalation of milnacipran would be 50 mg a day for about 3 days, followed by about 25 mg to 75 mg for about 3 days and then followed by a dosage of greater than 100 mg for a sufficient period of time to effectively treat the symptoms of the neurological disorder.

As another example, a preferable dosage regime for a 4-step dosage escalation of milnacipran would be about 20 mg to about 30 mg for about 7 days followed by administering a daily dosage of about 40 mg to about 60 mg for about 7 days followed by a daily dosage of about 75 mg to about 125 for about 7 days and lastly about 175 mg to about 225 for a sufficient period of time to effectively treat the symptoms of the neurological disorder.

Milnacipran is preferably administered greater than 100 mg/day and more preferably greater than 200 mg/day. In one embodiment, milnacipran is administered greater than about 100 mg/70 kg of body mass.

Divided Daily Dosage Amounts for Increased Daily Administration

Higher daily dosage amounts can be achieved by dividing the single daily dosage amount and administering it two or more times a day (e.g. 2, 3, 4, or 5). Any one or more of the anti-depressants, in the daily dosages described herein, can be administered in divided dosages, to obtain an effective serum drug concentration over an extended period of time.

For example, administering a daily dosage of about 200 mg of milnacipran can be administered about 100 mg twice-a-day (BID). Similarly administering a daily dosage of about 400 mg of milnacipran can be administered by about 200 mg twice-a-day (BID). This technique can be extended to administering higher dosage amounts of antidepressants:

Milnacipran can be administered between about 50 mg and 800 mg per day; preferably between about 100 mg and 400 mg per day and most preferably between about 200 mg and 300 mg per day.

Venlafaxine hydrochloride can be administered between about 75 mg and 1,500 mg per day; preferably between about 112.5 mg and about 900 mg per day and most preferably between about 225 mg and 600 mg per day.

Mirtazapine can be administered between about 15 mg and 180 mg per day; preferably between about 30 mg and about 120 mg per day and most preferably between about 45 mg and 60 mg per day.

Nefazodone hydrochloride can be administered between about 200 mg and 2,400 mg per day; preferably between about 300 mg and about 1200 mg per day and most preferably between about 600 mg and 800 mg per day.

Thioridazine hydrochloride can be administered between about 50 mg and 800 mg per day; preferably between about 100 mg and about 400 mg per day and most preferably between about 200 mg and 300 mg per day.

Bupropion hydrochloride can be administered between about 150 mg and 1,600 mg per day; preferably between about 300 mg and about 1200 mg per day and most preferably between about 400 mg and 600 mg per day.

Phenelzine sulfate can be administered between about 15 mg and 360 mg per day; preferably between about 60 mg and about 240 mg per day and most preferably between about 90 mg and 135 mg per day.

Tranylcypromine sulfate can be administered between about 30 mg and 160 mg per day; preferably between about 45 mg and about 120 mg per day and most preferably between about 60 mg and 80 mg per day.

Moclobeminde can be administered between about 400 mg and 3,600 mg per day; preferably between about 500 mg and about 2400 mg per day and most preferably between about 900 mg and 1200 mg per day.

Pirlindole can be administered between about 200 mg and 1,600 mg per day; preferably between about 300 mg and about 1,200 mg per day and most preferably between about 400 mg and 800 mg per day.

Citalopram hydrobromide can be administered between about 20 mg and 240 mg per day; preferably between about 40 mg and about 160 mg per day and most preferably between about 60 mg and 80 mg per day.

Paroxetine hydrochloride can be administered between about 20 mg and 250 mg per day; preferably between about 50 mg and about 200 mg per day and most preferably between about 62.5 mg and 80 mg per day.

Fluoxetine hydrochloride can be administered between about 20 mg and 320 mg per day; preferably between about 60 mg and about 240 mg per day and most preferably between about 80 mg and 120 mg per day.

Sertraline hydrochloride can be administered between about 25 mg and 800 mg per day; preferably between about 50 mg and about 200 mg per day and most preferably between about 75 mg and 100 mg per day.

Amitriptyline hydrochloride can be administered between about 50 mg and 600 mg per day; preferably between about 100 mg and about 400 mg per day and most preferably between about 150 mg and 200 mg per day.

Perphenazine and amitriptyline hydrochloride can be administered between about 6 mg and 75 mg respectively to 22 mg and 350 mg respectively; preferably between about 9 mg and 100 mg repectively to 15 mg and 200 mg respectively per day.

Desipramine hydrochloride can be administered between about 100 mg and 1200 mg per day; preferably between about 200 mg and 800 mg per day and most preferably between about 300 mg and 400 mg per day.

Doxepin hydrochloride can be administered between about 75 mg and 1200 mg per day; preferably between about 150 mg and 600 mg per day and most preferably between about 300 mg and 450 mg per day.

Trimipramine maleate can be administered between about 75 mg and 800 mg per day; preferably between about 150 mg and 600 mg per day and most preferably between about 200 mg and 300 mg per day.

The antidepressant can be provided in dosage formulations as currently approved for use, packaged with instructions to take over a period of time in the requisite number and intervals to reach the maintenance dosage, or in a dosage pack. For example, a dose pack of antidepressant may contain discrete dosages and instructions for taking the discrete dosages in increasing amounts over a time period until a maintenance dosage is reached. In one embodiment, the dosages are in the same amount and the instructions provide for taking an increased number of dosages over time. In another embodiment, the dose pack includes dosages containing different amounts of antidepressant and the instructions provide for taking the dosages in increasing amounts over time. Alternatively, the dose pack may be formulated to release an increasing amount of antidepressant over a period of days to reach a maintenance dosage, or the formulation may be a sustained release and/or pulsed released formulation.

A representatiave dose pack includes:

(a) a daily dosage of milnacipran of up to about 50 mg for more than about 3 days;

(b) a daily dosage of milnacipran of about 25 mg to about 75 mg for more than about 3 days; and

(c) a daily dosage of milnacipran of greater than about 100 mg for a sufficient period of time to effectively treat the symptoms.

Any patent, patent document, or reference disclosed herein is incorporated into reference into this invention and forms part of this invention.

### EXAMPLES

### Example 1

### Gradual or Dosing Escalation, and the Effect of Milnacipran for Treatment of Fibromyalgia.

This study was undertaken to characterize the efficacy of milnacipran in treatment of fibromyalgia syndrome (FMS). A secondary objective was to evaluate the relationship between daily dose administered, frequency of dosing, and efficacy in treatment of FMS. Anticipating that higher doses would be more effective, a third objective was to determine whether gradual escalation of dosage could increase the tolerated dose of milnacipran. In previous studies, patients were given initial daily dosages of milnacipran of 50 mg, 100 mg, or 200 mg, and the adverse event profile with 200 mg was substantially worse than with 100 mg or 50 mg. In this study, daily dosages were gradually escalated.

Methodology:

Subjects who met the 1990 ACR criteria for fibromyalgia syndrome were eligible for enrollment. Patients recorded baseline symptoms for the first two weeks after washing off anti-depressants, hypnotics and certain other drugs that potentially could interfere with efficacy measurements. Patients were randomized to a once daily milnacipran dose treatment group, a twice-daily milnacipran dose treatment group, or placebo control in a 1.5:1.5:1 ratio. Active treatment patients initially received 25 mg of milnacipran in one (25 mg QD) or two (12.5 mg BID) daily doses for the first week. If the patient tolerated this dosage, she was stepped up to a 50 mg daily dosage for week two, 100 mg for week three, and 200 mg for week four, or a matching placebo. If dose-limiting toxicities were encountered during any given week, the patient was stepped down to the previous week's dosage and maintained at that dosage for the remainder of the study. Subjects continued to take their maximum tolerated dose (up to 200 mg) for an additional 8 weeks. Patients may receive a total of 12 weeks of medication. Dose-limited toxicity is defined as the occurrence of a drug-related grade 3-4 adverse event.

Efficacy Measurements:

Efficacy was measured using FMS status assessments prior to receiving the first dose and during monthly clinic visits. These included the Fibromyalgia Impact Questionnaire, McGill Pain, patient clinical global impression, patient global pain status, the SF-36 quality of life measurement, and evoked pain measurements.

Results:

In previous studies, patients were escalated over a short time period (typically one week or less) to their final daily dosage. The 200 mg dosage gave a substantially worse rate of incidence of side-effects than the 50 mg or 100 mg dosages. Based on these results, it was expected in this study that approximately 50% of patients would be unable to tolerate the 200 mg daily dosage and would be treated with 100 mg/day. Surprisingly, most patients in the study went all the way to 200 mg/day and tolerated this dosage well. Out of approximately 70 treated with milnacipran, only 9 failed to tolerate 200 mg/day.

Side effects for patients given an unescalated initial dose of 100 mg milnacipran were worse in the first week of treatment and subsided to lower levels by weeks 2-4. This suggests that the slower escalation of dose was responsible for greater patient tolerance of a 200 mg daily dosage of milnacipran.

### Example 2

### Use of Milnacipran to Treat Fibromyalgia Syndrome METHODS:

A 12-week randomized, double-blind placebo-controlled dose escalation monotherapy trial was conducted to evaluate milnacipran in patients with a diagnosis of FMS. After an initial period when patients were washed off pain medication, centrally acting stimulants, antidepressants and sedative-hypnotics, a two-week baseline period was begun. After successful completion of the baseline period, patients were randomized to placebo, QD milnacipran, or BID milnacipran, in a ratio of 1:1.5:1.5. All patients were escalated over a 4-week period in weekly steps from 25 mg daily, to 50, 100, and finally 200 mg daily, or until dose-limiting toxicity (DLT) was evident. The presence of DLT was evaluated by the study center each week prior to authorizing the patient to step up to the next higher dosage. In the event that DLT was evident, the patient was stabilized at the previously well-tolerated dosage, and remained on this dose for eight weeks at stable dose therapy. Patients who successfully reached the 200 mg daily dose were also then treated for an additional 8 weeks at that dose.

At any give dose level, milnacipran QD patients would receive the full dose of milnacipran in the morning and receive placebo at night. Milnacipran BID patients would receive the same total amount in a split dose given morning and evening. During the study, patents were asked to carry an electronic diary and record pain, fatigue, sleep, and functional information. The custom designed diary captured spontaneous pain data in several ways, including

(1) random daily prompts (the device notified the patient to record their current level of pain 4-5 times per day);

(2) a morning daily prompt querying about the previous 24-hours' pain;

(3) a Friday night weekly report asking about the patient's average pain for the past week.

The electronic assessments were supplemented with traditional pain, mood, and quality of life inventories during clinic visits. The diaries implemented an electronic version of the Gracely anchored logarithmic pain scale. This scale asks patients to choose between “extremely intense,” “very intense,” “intense,” “strong,” “slightly intense,” “barely strong,” “moderate,” “mild,” “very mild,” “weak,” “very weak,” “faint,” and “no pain” sensation to describe their pain.

During the 4th, 8th, and 12th week of treatment, patients visited the clinic, where a number of standard outcome measures including the SF-36, the McGill pain questionnaire, the Beck, the STPI, the fibromyalgia impact questionnaire, and several pain measures were administered. Safety information was also collected at these clinic visits.

The primary endpoint was defined as the change in pain score from baseline to endpoint based on pain scores collected on the patient electronic diary (PED). Endpoint was defined as week twelve for assessments with a single value (such as clinic measures) or the average of scores at weeks 11 and 12 for diary-based outcomes. “Responders” were defined as those patients an improvement in pain score of at least 30%.

Table 1 shows the inclusion and exclusion criteria for the trial.

TABLE 1
Patient Criteria

| Inclusion Criteria Highlights | Exclusion Criteria Highlights |
|---|---|
| Diagnosed with primary fibromyalgia | Severe psychiatric illness as |
| as defined by the 1990 ACR. | determined by patient self-report |
| | on the screening exam. |
| Male or female between the ages of 18 | Significant risk of suicide. |
| and 70 years. | |
| Gracely intensity pain scale | Alcohol, benzodiazepene, or |
| responding (weekly recall) of at least | other drug abuse. |
| 10 or more on a 20 point scale at the | |
| end of baseline. | |
| Willing and able to use a PED device | Any history of behavior that |
| daily for minimum of 14 weeks. | would prohibit compliance for |
| | the duration of the study. |
| Willing to withdraw from CNS active | Any pending or current |
| therapies including anti-depressants, | disability claim, workman's |
| sedative-hypnotic agents, and centrally | compensation claim, or |
| acting analgesics. | litigation. |
| Only non-prescription doses of | |
| NSAIDs, aspirin, and acetaminophen | |
| for acute pain are allowed. | |

Results:

Table 2 shows the rate of dose escalation failures. The twice-daily dose of milnacipran produced a lower rate of dose intolerance than once daily dosing. This difference almost reached statistical significance, despite the small sample size (p=0.07). (“Blinded” in the table refers to patients whose treatment group at this date is still blinded to the investigators.)

TABLE 2
Dose Escalation Failures

| | | | % Dose |
|---|---|---|---|
| | Intolerance | Total Patients | Intolerance |
| MIL QD | 7 | 36 | 19% |
| MIL BID | 2 | 36 | 6% |
| Placebo | 0 | 23 | 0% |
| Blinded | 6 | 30 | 20% |

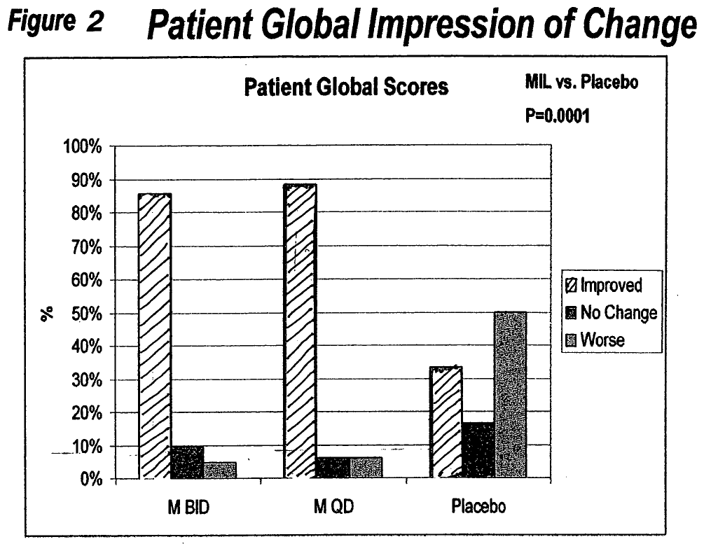

FIG. 2 presents perhaps the most striking evidence of milnacipran's efficacy. This figure plots patient global pain scores for all patients who reached endpoint at the time of this analysis (38 MIL and 12 placebo). On a 1-7 scale, where 1 is very much improved, 4 is unchanged, and 7 is very much worse, the mean value for MIL patents was 2.3, while the mean value for placebo patients was 4.3. In FIG. 2, scores of 1-3 are shown as improved, 4=no change, and 5-7 is worse. The difference between the milnacipran groups and placebo is statistically significant at p=0.0001.

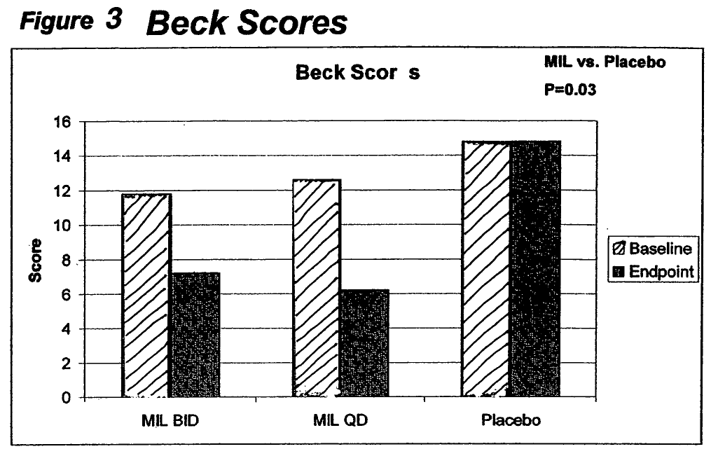

FIG. 3 indicates the change in Beck scores during the treatment phase of the trial. While this trial did not select for depressed patients, there is a fairly high rate of major depression and depressed mood in the FMS population. In this sample, there were 14 patients meeting the definition of MDE. The difference in scores at endpoint between the pooled milnacipran groups and the placebo group was significant at p=0.03, indicating that the known anti-depressant effects of MIL are perhaps an important adjunct in treating this population.

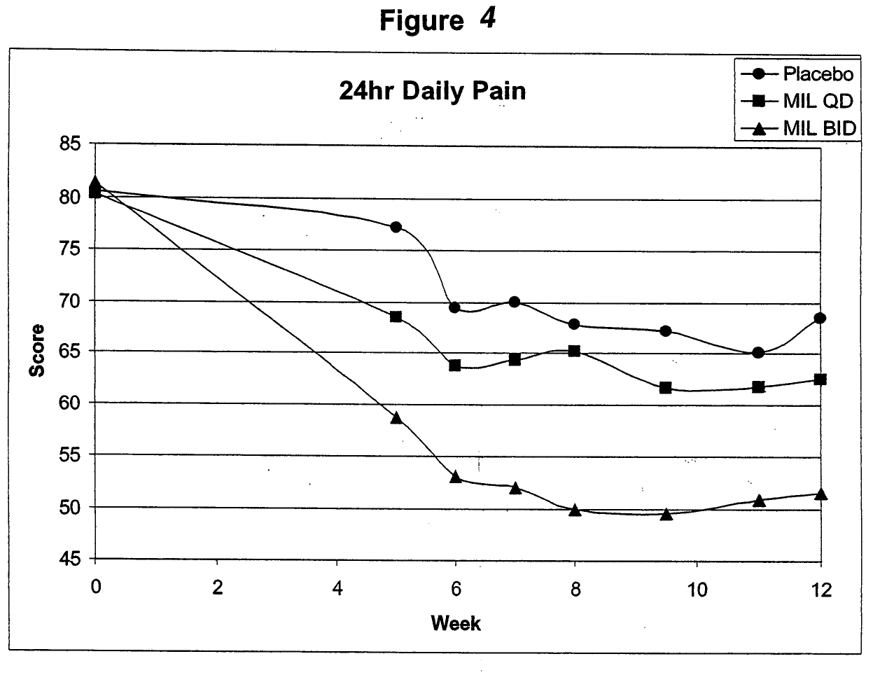

FIG. 4 shows 24-hour daily pain reported scores of the three treatment groups over the course of the study. BID dosing was more effective than QD dosing throughout the period of treatment.

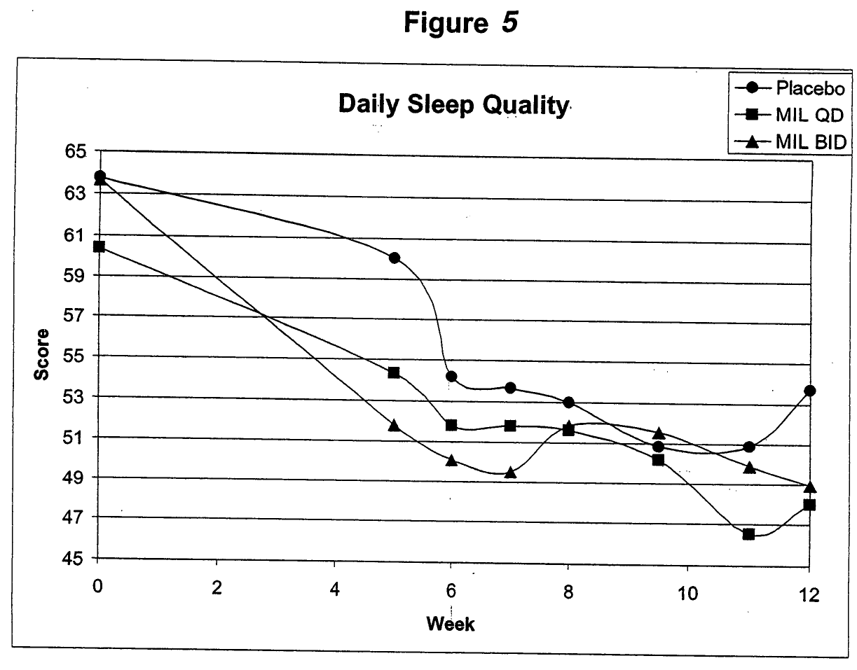

FIG. 5 shows the daily sleep quality scores reported by the patients in the three treatment groups over the course of the study. No significant improvement in sleep quality was found with milnacipran as compared to placebo, and no difference between QD and BID dosing was detected. On the other hand, no worsening of sleep quality was found, even with BID dosing, which involved administering a dose in the evening. This is notable, because a common side-effect of anti-depressants is interfering with sleep quality.

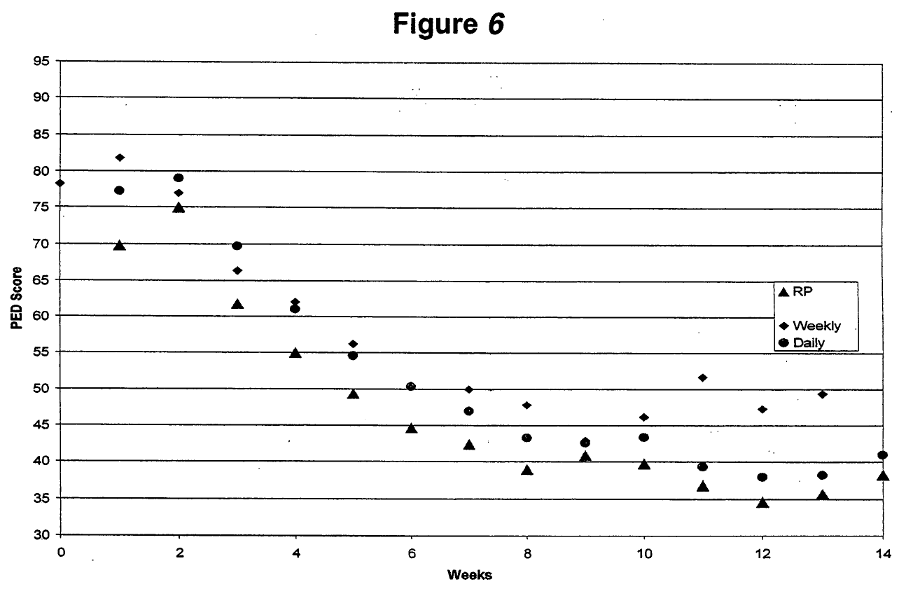

FIG. 6 shows the patient electronic diary (PED) pain scores of patients classified as “responders” who received milnacipran BID. The number of patients whose scores were averaged for each data point was 34 at week 1, declining to 24 at weeks 13 and 14. RP is the random prompt pain scores averaged over a week. Weekly is the weekly recall score. Daily is the daily recall score. Note that the first two weeks are the baseline period, weeks 3-6 the dose escalation period, and weeks 6-end the stable dose treatment period. The improvement in pain scores over weeks 2-6 with dose escalation suggests higher doses of milnacipran are more effective in treating pain associated with FMS.

Conclusions:

Milnacipran effectively treated pain associated with fibromaylgia syndrome. It also improved mood in depressed patients with FMS. The relatively high dose of 200 mg per day was used. The improvement in pain scores as the dose was escalated to 200 mg indicates this dose is important to the alleviation of pain. Twice-daily dosing was more effective than once daily dosing in reducing pain. Twice daily dosing also gave fewer dose-related adverse events and less intolerance to the increased dosages used than did once-daily dosing.

### Example 3

### Treatment with Milnacipran Reverses Swim Stress-Induced Muscle Hyperalgesia.

It has been shown that repeated inescapable swim stress produces a delayed and long-term cutaneous hyperalgesia to both thermal and prolonged chemical noxious stimuli in rats. This swim stress-induced hyperalgesia (SSIH) model shares some characteristics with FMS. These similarities include (1) the presence of cutaneous hyperalgesia, (2) a significant role of stress, (3) an involvement of NMDA receptor mechanisms in the hyperalgesic responses, and (4) antidepressant efficacy. Specifically, pretreatment with clormipramine and fluoxetine prevent the development of cutaneous hyperalgesia in repeatedly stressed rats, and certain antidepressants ameliorate pain and sleep abnormalities in FMS.

Methods:

Milnacipran (MIL) was mixed in normal saline and administered via intraperitoneal injection (IP) to Sprague-Dawley rats (200-300 g).

The treatment groups of rats were the following: (1) stressed—forced swimming in 20 cm of water; (2) sham—swimming in 2-3 cm of water; (3) naive—left undisturbed in cage. Rats were given no injection, saline IP QD, or milnacipran 10 or 30 mg/kg/day IP QD.

The rats were tested for thermal nociception threshold by hot plate response latency (in seconds). The rats were tested for muscle hyperalgesia by measuring grip strength (in kg) by algometer.

When testing the effect of pretreatment with milnacipran, the rats were given drug on days 1-11, and were subjected to swim stress on days 8-10. Data on hot plate response latency and grip strength was acquired on days 7 and 11. When testing the effect of post-treatment with milnacipran, hot plate response latency and grip strength were measured on days 1, 5, and 10; the swim stress was administered on days 2-4; and the drug treatment was administered on days 5-10.

Results:

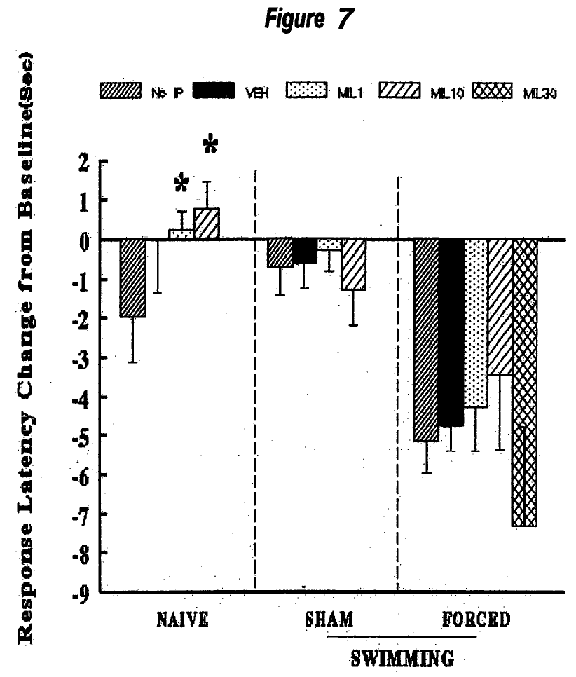

FIG. 7 shows that swim stress-induced reductions in hot plate latency, and that these reductions were not prevented by milnacipran pretreatment.

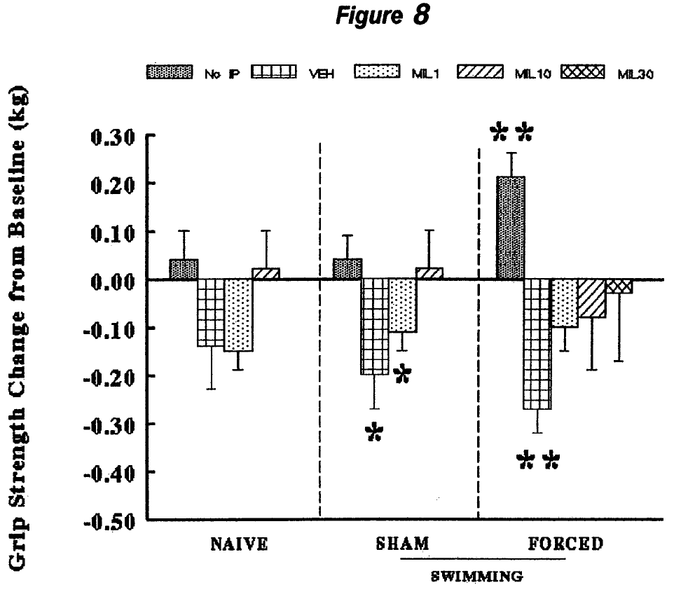

FIG. 8 shows that repeated stress (IP injection and forced/sham swimming) caused a reduction in grip strength. Pretreatment with milnacipran prevented this stress-induced reduction in grip strength. Thus, pretreatment with milnacipran prevented muscle hyperalgesia in this model of FMS.

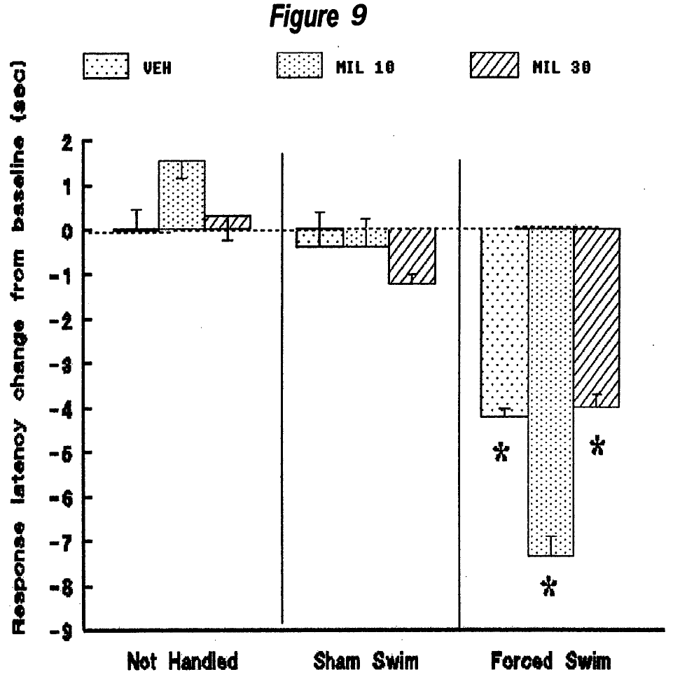

FIG. 9 shows that milnacipran treatment after the swim stress did not reverse swim stress-induced reductions in hot plate response latency.

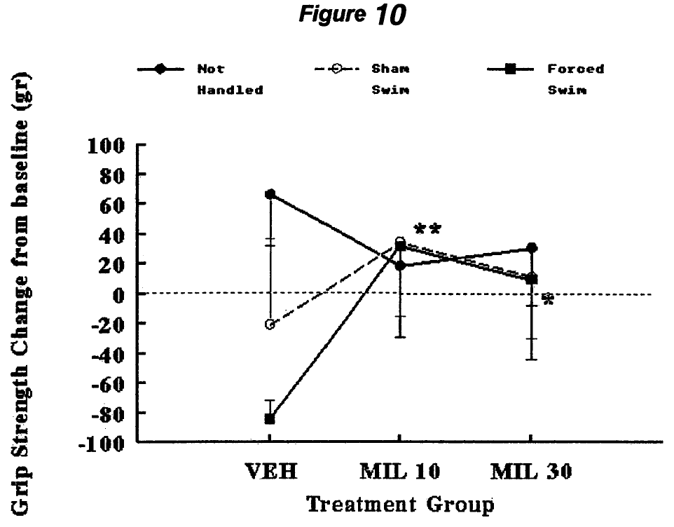

FIG. 10 shows, however, that milnacipran did reverse the reduction in grip strength caused by repeated forced swim stress.

Conclusions:

Thermal cutaneous hyperalgesia evoked by swim stress is persistent and remains essentially unchanged for several days post conditioning.

Swim stress followed by repeated IP injection induces grip strength weakening that appears to be associated with muscular allodynia.

Milnacipran is efficacious in reversing and preventing muscular allodynia caused by swim stress. However, milnacipran does not reverse or prevent cutaneous thermal hyperalgesia.

Modulation of cutaneous and muscular nociception can be dissociated in this animal model, since they can exist and be pharmacologically affected independently.

Numerous modifications and variations of the present invention are possible in light of the above teachings. It is therefore to be understood that within the scope of the appended claims, the invention may be practiced otherwise than as specifically described herein.

We claim:

1. A method of treating symptoms of a chronic neurological disorder or pain associated therewith in a patient in need of treatment comprising:
administering an antidepressant in an escalating dosage over a period of time until a maintenance dosage is reached.

2. The method of claim 1 wherein the chronic neurological disorder is selected from the group consisting of CFS, FMS, DSP, FSD, depression and pain.

3. The method of claim 1 wherein the chronic neurological disorder is fibromyalgia syndrome.

4. The method of claim 1 wherein the neurological disorder is chronic fatigue syndrome.

5. The method of claim 1 wherein the neurological disorder is pain.

6. The method of claim 1 wherein the antidepressant is selected from the group consisting of noradrenaline-serotonin reuptake inhibitors (NSRIs), selective serotonin reuptake inhibitors (SSRIs), monoamine oxidase inhibitors; anti-convulsants and non-specific anti-depressants.

7. The method of claim 1 wherein the antidepressant has analgesic properties.

8. The method of claim 1 where in the antidepressant has NMDA-antagonist properties.

9. The method of claim 1 wherein the antidepressant is milnacipran.

10. The method of claim 1 wherein the antidepressant is administered in increasing dosage amounts in a stepwise manner to increase the circulating dosage of the active compound to avoid or minimize side effects.

11. The method of claim 1 wherein the antidepressant is administered in an initial dosage amount of up to 100 mg per day for a first period of time and wherein a second dosage amount of approximately 1.5 to 2.5 times greater than the first dosage amount is administered for a second period of time to treat symptoms of the chronic neurological disorder.

12. The method of claim 11 further comprising administering a third dosage amount about 1.5 to 2.5 times greater than the second dosage amount for a third period of time to treat symptoms of the chronic neurological disorder.

13. The method of claim 12 further comprising administering a fourth dosage amount about 1.5 to 2.5 times greater than the third dosage amount for a fourth period of time to treat symptoms of the chronic neurological disorder.

14. The method of claim 10 wherein the time periods for each administration are greater than 3 days.

15. The method of claim 14 wherein the time periods for each administration are between 2 and 12 weeks.

16. The method of claim 1 wherein the antidepressant is administered in a dosage escalation regime providing a positive patient safety profile (e.g., dose limiting toxicity) and a suitable peak plasma concentration (Cₘₐₓ) of drug.

17. The method of claim 1 comprising providing a dose pack of antidepressant containing discrete dosages and instructions for taking the discrete dosages in increasing amounts over a time period until a maintenance dosage is reached.

18. The method of claim 17 wherein the dosage pack comprises milnacipran and the dosage is escalated over a period of days to reach the maintenance dosage.

19. The method of claim 1 providing antidepressant formulated to release an increasing amount of antidepressant over a period of days to reach a maintenance dosage.

20. The method of claim 19 wherein formulation is a sustained release and/or pulsed released formulation.

21. The method of claim 1 comprising:
(a) administering to a patient in need thereof a daily dosage of milnacipran of up to about 50 mg for more than about 3 days;
(b) administering to the patient a daily dosage of milnacipran of about 25 mg to about 75 mg for more than about 3 days; and
(c) administering to the patient a daily dosage of milnacipran of greater than about 100 mg for a sufficient period of time to effectively treat the symptoms.

22. A dose pack of antidepressant containing discrete dosages and instructions for taking the discrete dosages in increasing amounts over a time period until a maintenance dosage is reached.

23. The dose pack of claim 22 wherein the dosages are the same amount and the instructions provide for taking an increased number of dosages over time.

24. The dose pack of claim 22 wherein the dosages contain different amounts of antidepressant and the instructions provide for taking the dosages in increasing amounts over time.

25. The dose pack of claim 22 wherein the dosage pack comprises milnacipran and the dosage is escalated over a period of days to reach the maintenance dosage.

26. The dose pack of claim 22 providing antidepressant formulated to release an increasing amount of antidepressant over a period of days to reach a maintenance dosage.

27. The dose pack of claim 26 wherein the formulation is a sustained release and/or pulsed released formulation.

28. The dose pack of claim 22 comprising:
(a) a daily dosage of milnacipran of up to about 50 mg for more than about 3 days;
(b) a daily dosage of milnacipran of about 25 mg to about 75 mg for more than about 3 days; and
(c) a daily dosage of milnacipran of greater than about 100 mg for a sufficient period of time to effectively treat the symptoms.
